(19) United States
(12) Patent Application Publication
(10) Pub. No.: US 2006/0270026 A1
(43) Pub. Date: Nov. 30, 2006

(54) Low-Mass Thermal Cycling Block

(75) Inventors: Ui Leng Soh, Singapore (SG); Hock Lai Khoo, Singapore (SG)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: Applera Corporation, Foster City, CA (US)

(21) Appl. No.: 11/383,140

(22) Filed: May 12, 2006

Related U.S. Application Data
(60) Provisional application No. 60/680,891, filed on May 13, 2005.

(51) Int. Cl. C12P 19/34; B01L 3/00; C12M 1/34

(52) U.S. Cl. 435/303.1; 435/288.4; 422/102; 435/91.2; 435/809

(57) ABSTRACT

An apparatus and method for rapid thermal cycling including a thermal block manufactured by metal-injection molding (MIM). The thermal cycling of a biological product can be more rapidly achieved by including a MIM thermal block with a thermoelectric module thermally coupled to the thermal block, a heat sink thermally coupled to the thermoelectric module, and a resistive heater thermally coupled to the thermal block.

### CROSS REFERENCE TO RELATED APPLICATIONS

This application claims a priority benefit under 35 U.S.C. § 119(e) from U.S. Provisional Patent Application No. 60/680,891 filed May 13, 2005, which is incorporated herein by reference.

### FIELD

The present teachings relate to thermal cycling of biological samples. More specifically, the present teachings relate to low-mass thermal cycling blocks.

### INTRODUCTION

In the biological field, thermal cycling can be utilized to provide heating and cooling of reactants in a reaction vessel. Examples of reactions of biological samples include polymerase chain reaction (PCR) and other reactions such as ligase chain reaction, antibody binding reaction, oligonucleotide ligations assay, and hybridization assay. In PCR, biological samples can be thermally cycled through a temperature-time protocol that includes melting DNA into single strands, annealing primers to the single strands, and extending those primers to make new copies of double-stranded DNA. During thermal cycling, it is desirable to heat and cool a thermal block rapidly and uniformly. The present teachings provide low-mass thermal cycling blocks. Existing blocks are machined, the present teachings provide manufacturing methods for constructing low mass thermal cycling blocks for rapid and uniform temperatures between samples wells leading to uniform quantification of reaction results.

### SUMMARY

In various embodiments, the present teachings can provide an apparatus for thermally cycling biological samples including a thermal block for receiving the biological samples, a thermoelectric module coupled to the thermal block, and a heat sink, wherein said heat sink is coupled to said thermoelectric module, wherein the thermal block is produced by MIM.

In various embodiments, the present teachings can provide a method for thermally cycling biological sample including providing a thermal block, wherein the thermal block is produced by MIM process, heating the thermal block, and cooling the thermal block, wherein the heating and cooling provide substantial temperature uniformity throughout a plurality of biological samples contained in a plurality of sample wells.

In various embodiments, the present teachings can provide an apparatus for thermally cycling biological samples including means for receiving the biological samples, means for heating the biological samples, and means for cooling the biological samples, wherein the heating and cooling provide substantial temperature uniformity throughout a plurality of biological samples contained in a plurality of sample wells, wherein means for receiving the biological samples is produced by MIM.

It is to be understood that both the foregoing general description and the following description of various embodiments are exemplary and explanatory only and are not restrictive.

### BRIEF DESCRIPTION OF THE DRAWINGS

The accompanying drawings, which are incorporated in and constitute a part of this specification, illustrate various embodiments. In the drawings,

FIG. 1 illustrates a machined thermal cycler block;

FIG. 2A-2B illustrate a low-mass thermal cycling block according to the present teachings; and

FIG. 3 illustrates a low-mass thermal cycling block according to the present teachings.

### DESCRIPTION OF VARIOUS EMBODIMENTS

In this application, the use of the singular includes the plural unless specifically stated otherwise. In this application, the use of “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Wherever possible, the same reference numbers will be used throughout the drawings to refer to the same or like parts.

The section headings used herein are for organizational purposes only, and are not to be construed as limiting the subject matter described. All documents cited in this application, including, but not limited to patents, patent applications, articles, books, and treatises, are expressly incorporated by reference in their entirety for any purpose.

In various embodiments, the apparatus for thermally cycling biological samples provides heat-pumping into and out of a thermal block, resistive heating of the thermal block, and diffusive cooling of the thermal block. The term “thermal cycling” or grammatical variations of such as used herein refer to heating, cooling, temperature ramping up, and/or temperature ramping down. Thermal cycling during temperature ramping up, when heating the thermal block assembly above ambient (20° C.), can comprise resistive heating of the thermal block assembly and/or pumping heat into the thermal block assembly by the thermoelectric module against diffusion of heat away from the thermal block assembly. Thermal cycling during temperature ramping down, when cooling the thermal block assembly above ambient (20° C.), can comprise pumping heat out of the thermal block assembly by the thermoelectric module and diffusion of heat away from the thermal block assembly against resistive heating.

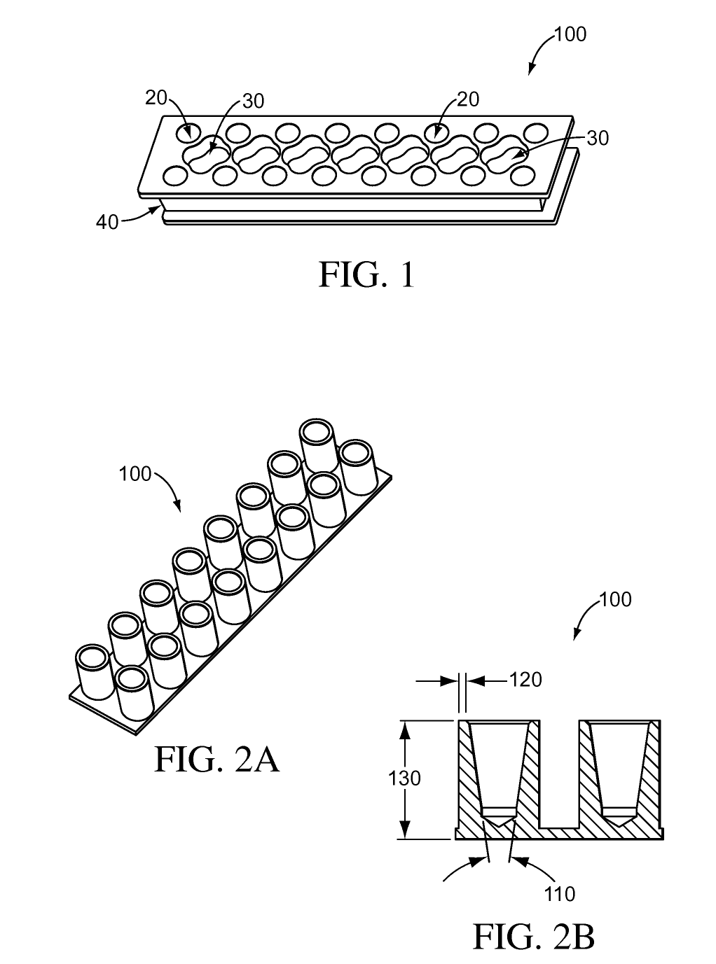

As illustrated in FIG. 1, thermal blocks have been either machined from a solid piece of metal or formed by coupling several pieces of metal together. As can be seen from the machined block 10, well-bores 20 form the recesses for wells that can contain samples for thermal cycling. Mass-reduction bores 30 remove mass from the block to reduce the thermal mass of the block 10. The sides 40 are machined to reduce the mass on the sides dimensions of the block. It is desirable to form a thermal block without machining.

In various embodiments, metal injection molding (MIM) can provide a process for manufacturing metal parts. MIM can combine the design freedom of plastic injection molding with the performance of metal. MIM can be used with metals such as aluminum, copper, tungsten, and alloys thereof.

In various embodiments, MIM can include feedstock mixing wherein very small powers are mixed with a thermoplastic polymer (known as a binder) to form a precise mixture of ingredients that is pelletized and directly fed into a plastic molding machine. This pelletized powder-polymer is known as feedstock. The metal powder and binders are mixed and heated in a mixer and cooled to form granulated feedstock. MIM can further include injection molding, wherein the feedstock is heated to melt the plastic and then with pressure is forced into a mold to form the desired geometry. The molded part is known as the “green” part. MIM can further include de-binding, wherein the polymer or binder is removed thermally by heating the “green” part to 400 degrees Celcius or 752 degrees Fahrenheit. While retaining its shape and size, the de-bound or “brown” part is a powder skeleton that is very brittle and porous. De-binding is performed in an oven where heat and air flow are fluxed in and exhaust products are fluxed out. The oven converts the “green” part to the “brown” part. MIM can further include sintering, wherein the “brown” part is heated to more than 1200 degrees Celcius or 2192 degrees Fahrenheit allowing densification and shrinking of the powder into a dense solid with the elimination of pores. Sintering is performed in an over where heat, hydrogen gas, and argon gas are fluxed in. Usually the sintering density is similar to a casting at about 98% of theoretical. The end result is the molded thermal part.

In various embodiments, MIM can provide parts with sizes of 100 millimeters by 100 millimeters. A typical 9-well thermal clock has larger dimensions. However, several block segments can be constructed by MIM to provide thermal cycling for a 96-well or 384-well consumable as described in European Pat. No. 1216098.

Reference will now be made to various embodiments, examples of which are illustrated in the accompanying drawings. Wherever possible, the same reference numbers are used in the drawings and the description to refer to the same or like parts.

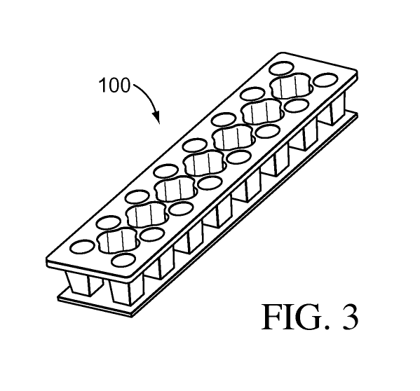

In various embodiments, as illustrated in FIGS. 2A and 2B, thermal block 100 can provide low thermal mass with minimum attachment points to each section encircling the sample well and minimizing the thickness of the wall in sections around the sample well. As illustrated in FIG. 3, thermal block 100 can provide different configurations configured to contain sample wells with reactions volumes ranging from 5.0 microliters to 100 microliters. In various embodiments, the sample block material can be copper, aluminum, or silver. Chart 1 below shows a comparison of the thermal blocks described above demonstrating the differences in temperature ramp rate in degrees Celcius per second for different materials.

CHART 1

| Thermal Block | | | |
|---|---|---|---|
| Material | | | |
| Copper | 5.53 | 9.3 | 8.43 |
| Aluminum | 7.71 | 12.91 | 11.82 |
| Silver | 7.81 | 13.14 | 11.94 |

In various embodiments, the thermal blocks illustrated in FIGS. 2-3 can be manufactured by MIM. The thermal blocks in FIGS. 2-3 can be used in an apparatus for thermally cycling biological samples. The thermal blocks can be adapted to receiving the biological samples. A thermoelectric module can be coupled to the thermal block with a heat sink underneath to provide radiation of the heat pumped from the thermal block. In various embodiments, the thermal block can include several block segments as shown in FIGS. 2-3 having, for example, capable of receiving two rows of sample wells. In various embodiments, the thermal block can include copper, silver, aluminum, and/or gold. It is desirable that the thermal block provide substantial temperature uniformity throughout the array of biological samples contained in the array of sample wells coupled to the block for thermal cycling.

In various embodiments, the apparatus for thermally cycling biological samples can further include a resistive heater positioned around the periphery of the thermal block and/or the periphery of the holes adapted to receive the sample wells such that the thermal block provides substantial temperature uniformity to the biological samples in the sample wells by distributing heat provided by the resistive heater. In various embodiments, the thermoelectric module can be used to provide biased heating to the thermal block during heating cycles and to remove heat by pumping it out of the thermal block.

In various embodiments, methods for thermally cycling biological sample can be provided by the present teachings by providing the thermal block produced by a MIM process such that heating and cooling of the thermal block provides substantial temperature uniformity throughout the plurality of biological samples contained in the plurality of sample wells. The heating can be provided by heat from a resistive heater. In various embodiments, the cooling can be provided by pumping heat out with the thermoelectric module, which can be also be used for providing bias heat during heating cycles. In various embodiments, the cooling can be provided by spinning the block thereby convectively dissipating heat from the thermal block to the environment during cooling cycles. For example, the thermal block can be disk-like in shape and provide concentric rings of holes to receive the sample wells. The disk can spin along the central axis creating a convective current over the thermal block. Alternatively, the thermal block of any shape can spin along an axis balanced by another thermal block to provide a convective current similar to a centrifuge. In various embodiments, cooling can be achieved by providing forced gas, such as air or nitrogen, to contact the thermal block. The forced gas can have ambient temperature or can be chilled to below ambient temperature.

In various embodiments, MIM can provide thermal blocks that cannot be produced by machining the thermal block from a solid piece of metal because the MIM thermal block has a thickness that cannot be uniformly machined such that every one of the plurality of sample wells is surrounded by portions of the thermal block having similar thickness. For example, MIM can provide rounded surfaces for contacting the sample wells and rounded exterior surfaces with a flat bottom as in FIGS. 2A and 2B. Alternatively, MIM can provide more than one exterior surface for rigidity while removing interior material other than rounded surfaces for contacting the sample wells as in FIG. 3. Also, MIM can provide multiple segments of thermal blocks more rapidly and consistently than machining.

In various embodiments, MIM can provide thermal blocks that can have temperature uniformity sufficient to avoid using resistive edge heating and providing heating and cooling using thermoelectric modules only. Examples of such performance are described in the example below.

### EXAMPLE

The following lists the conditions for the thermal blocks tested. Block material: copper block with thermal pad, no resistive edge heater, no copper insert in the heat sink, TEC set current +4.5 A & −5.5 A, sample volume 10 uL, adjacent test strips temperature differential 25° C., heated cover temperature 105° C., empty tray consumable, heated cover pressure of 6kg dead weight. A first and second test consisted of six test strips. The results of the first test provided averages of 3.68° C./sec peak bulk heat rate (standard deviation 0.0753), −4.97° C./sec peak bulk cool rate (standard deviation 0.1633), and 2.79° C./sec effective ramp rate (standard deviation 0.0354), were measured over the six test strips. The results of the second test provided averages of 3.60° C./sec peak bulk heat rate (standard deviation 0.0632), −4.88° C./sec peak bulk cool rate (standard deviation 0.0753), and 2.76° C./sec effective ramp rate (standard deviation 0.0242), were measured over the six test strips. The first test provided for 10 uL volumes provided a temperature nonuniformity for ramping up temperature for 10 seconds of 0.401-0.724° C. The second test provided for 10 uL volumes provided a temperature nonuniformity for ramping up temperature for 10 seconds of 0.364-0.776° C. The first test provided for 10 uL volumes provided a temperature nonuniformity for ramping down temperature for 10 seconds of 0.132-0.306° C. The second test provided for 10 uL volumes provided a temperature nonuniformity for ramping down temperature for 10 seconds of 0.112-0.278° C. The first test provided for 50 uL volumes provided a temperature nonuniformity for ramping up temperature for 10 seconds of 0.221-0.603 ° C. The second test provided for 50 uL volumes provided a temperature nonuniformity for ramping up temperature for 10 seconds of 0.172-396 ° C. The first test provided for 50 uL volumes provided a temperature nonuniformity for ramping down temperature for 10 seconds of 0.177-0.727° C. The second test provided for 50 uL volumes provided a temperature nonuniformity for ramping down temperature for 10 seconds of 0.197-0.386° C.

For the purposes of this specification and appended claims, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all subranges subsumed therein. For example, a range of “less than 10” includes any and all subranges between (and including) the minimum value of zero and the maximum value of 10, that is, any and all subranges having a minimum value of equal to or greater than zero and a maximum value of equal to or less than 10, e.g., 1 to 5.

It is noted that, as used in this specification and the appended claims, the singular forms “a,” “an,” and “the,” include plural referents unless expressly and unequivocally limited to one referent. Thus, for example, reference to “a thermoelectric module” includes two or more thermoelectric modules.

It will be apparent to those skilled in the art that various modifications and variations can be made to various embodiments described herein without departing from the spirit or scope of the present teachings. Thus, it is intended that the various embodiments described herein cover other modifications and variations within the scope of the appended claims and their equivalents.

What is claimed is:

1. An apparatus for thermally cycling biological samples, the apparatus comprising:
a thermal block for receiving the biological samples;
a thermoelectric module thermally coupled to the thermal block; and
a heat sink, wherein said heat sink is thermally coupled to the thermoelectric module,
wherein the thermal block is produced by MIM.

2. The apparatus of claim 1, wherein the thermal block comprises several block segments.

3. The apparatus of claim 1, wherein the thermal block comprises at least one of copper, silver, aluminum, and gold.

4. The apparatus of claim 1, wherein the thermal block is adapted to provide substantial temperature uniformity throughout an array of biological samples contained in an array of sample wells.

5. The apparatus of claim 4, further comprising a resistive heater, wherein the thermal block provides substantial temperature uniformity by distributing heat provided by the resistive heater.

6. The apparatus of claim 5, wherein the thermal block provides substantial temperature uniformity by distributing heat provided by the thermoelectric module.

7. The apparatus of claim 4, wherein the thermal block provides substantial temperature uniformity by removing heat pumped out by the thermoelectric module.

8. A method for thermally cycling biological sample, the method comprising:
providing a thermal block, wherein the thermal block is produced by MIM process;
heating the thermal block; and
cooling the thermal block;
wherein the heating and cooling provide substantial temperature uniformity throughout a plurality of biological samples contained in a plurality of sample wells.

9. The method of claim 8, wherein heating comprises providing heat from a resistive heater.

10. The method of claim 9, wherein cooling comprises pumping heat out with the thermoelectric module.

11. The method of claim 10, wherein heating further comprises providing heat from a thermoelectric module.

12. The method of claim 9, wherein cooling comprises spinning the block to provide convective dissipation of heat to the environment.

13. The method of claim 9, wherein cooling comprises providing forced gas and contacting the forced gas with the thermal block.

14. The method of claim 13, further comprising cooling the forced gas below ambient temperature.

15. An apparatus for thermally cycling biological samples, the apparatus comprising:
means for receiving the biological samples;
means for heating the biological samples; and
means for cooling the biological samples,
wherein the heating and cooling provide substantial temperature uniformity throughout a plurality of biological samples contained in a plurality of sample wells,
wherein means for receiving the biological samples is produced by MIM.

16. The apparatus of claim 15, wherein the means for receiving the biological samples is a thermal block produced by MIM that cannot be produced by machining the thermal block from a solid piece of metal.

17. The apparatus of claim 16, wherein the thermal block has a thickness, wherein the thickness cannot be uniformly machined such that every one of the plurality of sample wells is surrounded by portions of the thermal block having similar thickness.

18. The apparatus of claim 15, wherein the MIM provides rounded surfaces for contacting the sample wells and rounded exterior surfaces with a flat bottom.

19. The apparatus of claim 15, wherein the MIM provides more than one exterior surface for rigidity and removes interior material other than rounded surfaces for contacting the sample wells.

20. The apparatus of claim 15, wherein the MIM provides portions of the means for receiving the biological samples.
